(19) United States
(12) Patent Application Publication
(10) Pub. No.: US 2014/0186465 A1
(43) Pub. Date: Jul. 3, 2014

(54) LOTION FOR CLEANING HUMAN ANORECTAL AREA

(71) Applicant: William Lei, Orleans (CA)

(72) Inventor: William Lei, Orleans (CA)

(21) Appl. No.: 14/196,255

(22) Filed: Mar. 4, 2014

Related U.S. Application Data
(63) Continuation of application No. 13/503,201, filed on Oct. 18, 2012.

(30) Foreign Application Priority Data
Oct. 20, 2009 (CA) 2683258

(51) Int. Cl. A61K 36/886; A61K 35/02; A61K 33/30

(52) U.S. Cl. CPC A61K 36/886; A61K 33/30; A61K 35/02
USPC 424/642

(57) ABSTRACT

A cleansing lotion composition of zinc derivatives, aloe vera, lanolin and a mixture of water-based emulsifying agents for reducing itchiness, promoting healing and reducing inflammation around the anorectal area of a human subject is disclosed. The subject conveniently spreads a suitable amount of the cleansing lotion placed on toilet paper and applies to the affected anorectal area. The excess lotion could be wiped clean with fresh toilet paper after each application.

### FIELD OF THE INVENTION

The present invention relates to cleaning human anorectal area and, more particularly, to a cleaning lotion for application in these areas.

### BACKGROUND OF THE INVENTION

Anorectal disorder, such as hemorrhoid, fistula and fissure causes much discomfort and posts health hazards. While some such disorders can be surgically treated, regular cleanings of the affected rectal wound area are critical in promoting speedy healing and preventing future recurrence of the disorder.

The inventor suffered from rectal fistula and an operation was preformed to attempt to treat the condition. However, the symptoms recurred in a different location a few months after the surgery. The inventor decided to refrain from having a second surgery as the disease could still return in yet another location. In addition, it is known that fistula removal surgery may result in the muscles in and around the rectal area being weaken, causing bowel control problem at an older age. Instead, for the past few years, the inventor took extreme care in personal hygiene to ensure the wound would not inflame or become worsen by washing the wound clean with soap and water following every bowel movement. It required the inventor great dexterity, patience, efforts and time in order to thoroughly clean and dry the affected rectal area. In addition, when the inventor was travelling or working away from home, it was evidently inconvenient to repeat the water/soap/drying routine.

It is therefore highly desirable to eliminate the need to clean the affected rectal area with the conventional water/soap/drying method and yet be able to ensure thorough cleanliness of the rectal area. The inventor tried to look for a cleansing lotion at pharmacies for this purpose but unfortunately nothing was available.

There exist some patents for topically treating anorectal disorders. For example, U.S. Pat. No. 4,945,084 (Packman) teaches compositions containing disaccharide polysulfate-aluminum compounds and other antifungal, anti-acne agents. In U.S. Pat. No. 6,242,010 (Hersh), a synergistic combination of reduced glutahione and selenium composition was disclosed. One of the manufacturing steps disclosed in published U.S. Patent Application No. US 2007/0042051 (Lim) requires heating. Thus, many of these prior art either require costly ingredients or too complicated to manufacture and some may also require prescriptions, thus making them less accessible.

U.S. Pat. No. 6,846,499 (El Mogy) teaches the use of Jojoba oil as lubricating, moisturizing and soothing agent for skin application. However, the therapeutic effect of such agent is unknown. Lastly, U.S. Pat. No. 6,844,356 (Do-Yoo) discloses a water solution of a mixture of quinine and camphor being injected into hemorrhoidal tissue for treating hemorrhoid. Such treatment involves injection and, as indicated in the disclosure therein, is not effective for anal fistula. Likewise, U.S. Pat. No. 5,554,370 (Uehara et al.) teaches a spherical activated carbon for treating patients with inflammatory bowel diseases which requires oral administration.

Thus, it is highly desirable to provide a simple yet effective cleaning lotion for topical use by patients with anorectal conditions. It was with this objective that the inventor employed his knowledge as a chemist to experiment by mixing different ingredients together and formulated his own cleansing lotion to wipe clean the fistula wound after bowel movements.

### List of Prior Art Literatures

Agren M S (1990), Studies on zinc in wound healing. (stockh) 154:1-36. PMID: 2275309
Barnes T 1947: The healing action of extracts of Aloe Vera on abrasions of the human skin. Amer. J. Botany 34.
Davis R H; Lettner M G; Russo J M; Byrne M E 1989: Wound healing. Oral & topical activity of Aloe Vera. J. Amer. Podiatric Med. Assoc. 79 (11) November, 559-562.
Davis R H; Leitmer M G; Russo J M 1988 Aloe Vera: A natural approach for treating wounds, oedema and pain in Diabetes. J. of the American Podiatric Medical Assoc. 78 (2) 60-68.

### SUMMARY OF THE INVENTION

Before explaining the disclosed embodiment of the present invention in detail it is to be understood that the invention is not limited in its application to the details of the particular arrangement shown since the invention is capable of other embodiments. Also, the terminology used herein is for the purpose of description and not of limitation.

It is an object of the present invention to provide a anorectal area cleansing lotion which will effectively sooth, clean and apply therapeutic effects for external, internal anorectal disorder, such as hemorrhoid, fissure or fistula.

It is another object of the present invention to provide such a lotion that is inexpensive and devoid of unintended medical side effects when applied on the patient's affected area.

It is yet another object of the present invention to have such a lotion available over-the-counter, i.e., without the need for prescription.

Accordingly, the present invention provides for a lotion for cleaning human anorectal area, consisting essentially of approximately 5 to 20% by volume of zinc derivatives, 1 to 5% by volume of unheated aloe vera, 0.2 to 1.8% by volume of anhydrous lanolin and a suitable amount of a mixture of water-based emulsifying agents.

The present invention further provides for a lotion for cleaning human anorectal area, consisting essentially of 10% by volume of zinc derivatives, 3% by volume of unheated aloe vera, 0.5% by volume of anhydrous lanolin and a suitable amount of a mixture of water-based emulsifying agents.

These and other objects and features of the present invention will become more readily apparent upon reference to the following detailed description of a preferred embodiment.

### DETAILED DESCRIPTION OF THE INVENTION

The inventor carried out numerous experimentations and trials for the lotion. In the first few months the wound became inflamed because his concoctions contained the wrong ingredients and composition, mostly with oil-based mixtures. At one point, the inventor had to stop the experimentations and revert to using the soap/water/dry routine. However, the inventor did not give up and experimented less with oil-based lotions and started mixing the ingredients again. After many months of trials, the inventor came up with a suitable cleansing lotion that required no more soap and water, successfully cleaning the wound after bowel movements. The lotion was suitably formulated; even today, the inventor's rectal fistula symptoms have disappeared and the problem has never returned. However, the inventor still uses his own formulated lotion on a daily basis to enhance the therapeutic effect on the affected anorectal area.

The lotion of the present invention reduces itchiness and promotes healing and possibly reduces inflammation around the anorectal area of a patient. The lotion consists of a composition made up essentially of zinc derivatives, aloe vera, lanolin and a mixture of emulsifying agents.

A preferred embodiment of the lotion comprises an effective amount of:
Zinc derivates, in the range of 5 to 20% by volume;
Unheated aloe vera, in the range of 1 to 5% by volume; and
Anhydrous lanolin, in the range of 0.2 to 1.8% by volume

A further preferred embodiment of the lotion comprises an effective amount of:
Zinc derivates, 10% by volume;
Unheated aloe vera, 3% by volume; and
Anhydrous lanolin, 0.5% by volume

Suitable non-medical carrier and water-based emulsifier and thickening agents are added to both embodiments of the lotion. Such emulsifier and thickening agents include stearic acid, glycol stearate, lecithin and/or xanthan gum, carboxymethyl-cellulose and glycerine could be used.

Optionally, a small and suitable amount of eucalyptus oil, clove oil, Jojoba oil or lavender oil could be used as an additive to enhance the fragrance of the cleansing lotion and also soothes the wound.

Alcohol and de-ionized water may also be added to the lotion composition.

In use, the patient spreads a suitable amount of the cleansing lotion placed on toilet paper and applies to the affected rectal area. The excess lotion could be wiped clean with fresh toilet paper after each application.

The inventor sought out other patients on whom to test the lotion of the present invention. The results follow:
Case 1: 52-year-old female suffering from external hemorrhoid for many years. A sit-bath was used to sooth the wound and to cleanse the area after each bowel movement but the subject found it very inconvenient. After using the cleansing lotion on toilet paper for a week, subject found it effective in terms of cleaning the area. Subject now uses less sit-bath to treat the area, resulting in much time saving.
Case 2: Female with a 13-month old daughter uses the cleansing lotion with toilet paper to wipe clean the soil when changing her daughter's diaper. Prior to using the instant cleansing lotion, subject routinely used a diaper wipe and applied zinc oxide to prevent diaper rash on her baby daughter. With the lotion, she now just cleans in one step.
Case 3: Middle aged male with copious amounts of hair in the anal region always requires an inordinate amount of time to adequately clean his rectal area. Subject finds that the combination of lotion on toilet paper is an effective means of cleaning the buttocks area.

Although it is not fully understood as to the working of this lotion is so dramatically effective in reducing itchiness, promoting healing and reducing inflammation around the rectal area of a patient, it is thought that the constituent ingredients have a synergistic effect when used in combination. The ingredients in this lotion are thought to play the following roles: The zinc derivatives, is an antibacterial agent and effectively reduce inflammation; the aloe vera also promotes healing in opened wound while lanolin also acts as a healing power agent in skin wound. The specific proportions of the ingredients is important.

While only preferred embodiments have been described and shown herein, obvious modifications are contemplated within the scope of this invention, as defined by the following claims.

1-13. (canceled)

14. A lotion for cleaning the human anorectal area, consisting of approximately 5-20% by volume of zinc derivatives, 1-5% by volume of aloe vera, 0.2-1.8% by volume of anhydrous lanolin, an optional fragrance oil, a mixture of emulsifying agents, and optionally a solvent selected from the group consisting of alcohols, deionized water, and mixtures thereof.

15. The lotion of claim 14, consisting of approximately 10% by volume of zinc derivatives, 3% by volume of aloe vera, 0.5% by volume of anhydrous lanolin, a fragrance oil, a mixture of emulsifying agents, and optionally a solvent selected from the group consisting of alcohols, deionized water, and mixtures thereof.

16. The lotion of claim 14, wherein said zinc derivatives is zinc oxide.

17. The lotion of claim 15, wherein said zinc derivatives is zinc oxide.

18. The lotion of claim 14, wherein said emulsifying agents are stearic acid, glycol stearate, lecithin, or a mixture thereof.

19. The lotion of claim 15, wherein said emulsifying agents are stearic acid, glycol stearate, lecithin, or a mixture thereof.

20. The lotion of claim 14, further comprising a fragrance oil that is eucalyptus oil, clove oil, Jojoba oil, or lavender oil.

21. The lotion of claim 15, wherein the fragrance oil is eucalyptus oil, clove oil, Jojoba oil, or lavender oil.

22. The lotion of claim 14, further comprising alcohol.

23. The lotion of claim 15, further comprising alcohol.

24. The lotion of claim 14, further comprising de-ionized water.

25. The lotion of claim 15, further comprising de-ionized water.

26. A method for soothing an internal or external anorectal disorder comprising, applying the lotion of claim 14 to the affected area of a person in need of such treatment.

27. The method of claim 26, wherein the internal or external anorectal disorder condition is a hemorrhoid, fissure, or fistula.

28. A method for soothing an internal or external anorectal disorder comprising, applying the lotion of claim 15 to the affected area of a person in need of such treatment.

29. The method of claim 28, wherein the internal or external anorectal disorder condition is a hemorrhoid, fissure, or fistula.

30. A method for cleaning the human anorectal area, comprising applying the lotion claim 14 to the anorectal area of a person in need of such treatment.

31. A method for cleaning the human anorectal area, comprising applying the lotion claim 15 to the anorectal area of a person in need of such treatment.
